(19) United States
(12) Patent Application Publication
(10) Pub. No.: US 2026/0069183 A1
(43) Pub. Date: Mar. 12, 2026

(54) DRIVING ABILITY DIAGNOSIS DEVICE, DRIVING ABILITY DIAGNOSIS METHOD, AND NON-TRANSITORY STORAGE MEDIUM

(71) Applicant: NeU Corporation, Tokyo (JP)

(72) Inventors: Ryuta KAWASHIMA, Tokyo (JP); Takeshi HOSHINO, Tokyo (JP); Masanori SHOJI, Tokyo (JP)

(21) Appl. No.: 19/320,022

(22) Filed: Sep. 5, 2025

(30) Foreign Application Priority Data
Sep. 6, 2024 (JP) 2024-154215
Aug. 7, 2025 (JP) 2025-132336

(51) Int. Cl. A61B 5/18; A61B 5/16; G06F 3/0488

(52) U.S. Cl. CPC A61B 5/18; A61B 5/162; G06F 3/0488

(57) ABSTRACT

A driving ability diagnosis device includes an output unit comprised to display a screen including a central region and a peripheral region, an input unit comprised to receive input from a user, and a controller comprised to cause the output unit to display a first mark or a second mark in the central region or the peripheral region a predetermined number of times for a predetermined period of time, judge whether input is received by the input unit while the first mark or the second mark is displayed, measure a reaction time that is a period of time from when the first mark is displayed to when the input unit receives input, and calculate scores for a plurality of items related to driving ability of the user, based on whether input from the user is received by the input unit and the reaction time.

### CROSS-REFERENCE TO RELATED APPLICATION

The present application claims priority from Japanese applications JP2024-154215, filed on Sep. 6, 2024, and JP2025-132336 filed Aug. 7, 2025, the contents of which are hereby incorporated by reference into this application.

### BACKGROUND

### Technical Field

The present invention relates to a driving ability diagnosis device, a driving ability diagnosis method, and a non-transitory storage medium.

### Description of the Related Art

There is a device for measuring a driving ability of a driver who drives a car (Non-Patent Literature 1). This device enables measurement of cognitive function items related to driving ability, such as processing speed, attention, and judgment. The driver is able to grasp his/her driving ability using the device.
[Patent Literature 1] Japanese Patent Laid-Open No. 2013-130734
[Non-Patent Literature 1] Rui Nouchi, Akiko Kobayashi, Haruka Nouchi and Ryuta Kawashima, “Newly Developed TV-Based Cognitive Training Games Improve Car Driving Skills, Cognitive Functions, and Mood in Healthy Older Adults: Evidence From a Randomized Controlled Trial,” Frontiers in Aging Neuroscience, May 2019, Volume 11, Article 99, Pages 1-15.

### SUMMARY

However, with the device in Non-Patent Literature 1, to measure each item related to a driving ability, such as cognitive processing speed, attention, judgment and the like of a driver, the driver is tested on a per-item basis. Accordingly, measurement of the driving ability takes a very long time (for example, from several to several tens of minutes). Accordingly, with the device, it is difficult to frequently measure the driving ability of a driver. The driving ability of a driver may constantly change depending on health conditions and the like.

Therefore, it is required to easily measure a plurality of items related to the driving ability, such as cognitive processing speed, attention, judgment and the like of a driver, in a short time of about one minute.

The present invention is aimed at providing a device that allows a plurality of items related to a driving ability to be easily measured.

To solve the problem mentioned above, the following means are adopted.

A first aspect is a driving ability diagnosis device including:
an output unit comprised to display a screen including a central region including a center position and a peripheral region other than the central region;
an input unit comprised to receive input from a user; and
a controller comprised to cause the output unit to display a first mark or a second mark different from the first mark in the central region or the peripheral region a predetermined number of times for a predetermined period of time, judge whether input from the user is received by the input unit while the first mark or the second mark is displayed, measure a reaction time that is a period of time from when the first mark is displayed to when the input unit receives input from the user, in a case where the first mark is displayed, and calculate scores for a plurality of items related to driving ability of the user, based on whether input from the user is received by the input unit while the first mark or the second mark is displayed and the reaction time.

An aspect of the disclosure may be attained through execution of a program by an information processing apparatus. That is, a configuration of the disclosure may be specified as a program for causing the information processing apparatus to execute processes to be executed by respective means in the aspect described above, or as a computer-readable recording medium storing the program. The configuration of the disclosure may also be specified as a method by which the information processing apparatus executes the processes to be executed by the respective means described above. The configuration of the disclosure may further be specified as a system including the information processing apparatus that executes the processes to be executed by the respective means described above.

Steps describing a program include not only processes that are performed chronologically in the described order, but also processes that are not necessarily performed chronologically but are performed in parallel or individually. Some steps describing the program may be omitted.

According to the present invention, there can be provided a device that allows a plurality of cognitive function items related to a driving ability to be easily measured.

### BRIEF DESCRIPTION OF THE DRAWINGS

FIG. 1 is a diagram illustrating an example functional block of a driving ability diagnosis device according to a first embodiment;

FIG. 2 is a diagram illustrating an example hardware configuration of an information processing apparatus;

FIG. 3 is a diagram illustrating an example operation of the driving ability diagnosis device according to the first embodiment;

FIG. 4 is a diagram illustrating an example of a screen at a time of start of a test by the driving ability diagnosis device according to the first embodiment;

FIG. 5 is a diagram illustrating a first example of the screen at the time of the test;

FIG. 6 is a diagram illustrating a second example of the screen at the time of the test;

FIG. 7 is a diagram illustrating an example of a radar chart indicating a score for each item related to a driving ability;

FIG. 8 is a diagram illustrating an example where a driving ability diagnosis device according to a second embodiment displays a character of a driver on a display device of an output unit;

FIG. 9 is a diagram illustrating an example operation of the driving ability diagnosis device according to the second embodiment;

FIG. 10 is a diagram illustrating an example of a character data table;

FIG. 11 is a diagram illustrating a specific example of the character data table;

FIG. 12 is a diagram illustrating an example of a character data table where scores for items are classified into a different number of categories than in FIG. 10;

FIG. 13 is a diagram illustrating an example of a two-dimensional chart where a relationship between a value on each axis and a change in each item is different than in FIG. 8;

FIG. 14 is a diagram illustrating a specific example of a character data table created according to inverted scores;

FIG. 15 is a diagram illustrating an example where “central attention” and “speed of cognition” are selected as a combination of two items;

FIG. 16 is a diagram illustrating a specific example of the character data table where “central attention” and “speed of cognition” are selected and scores are inverted;

FIG. 17 is a diagram illustrating an example where “peripheral attention” and “central attention” are selected as a combination of two items;

FIG. 18 is a diagram illustrating a specific example of the character data table where “peripheral attention” and “central attention”are selected and scores are inverted;

FIG. 19 is a diagram illustrating an example where “peripheral judgment” and “central judgment” are selected as a combination of two items;

FIG. 20 is a diagram illustrating a specific example of the character data table where “peripheral judgment” and “central judgment”are selected and scores are inverted;

FIG. 21 is a diagram illustrating an example where “peripheral concentration” and “central concentration” are selected as a combination of two items; and

FIG. 22 is a diagram illustrating a specific example of the character data table where “peripheral concentration” and “central concentration” are selected and scores are inverted.

### DESCRIPTION OF THE EMBODIMENTS

Hereinafter, embodiments will be described with reference to the drawings. Configurations of the embodiments are merely examples, and configurations of the invention are not limited to specific configurations of the disclosed embodiments.

To implement the invention, a specific configuration according to an embodiment may be adopted as appropriate.

### First Embodiment

### (Outline)

Here, a driving ability diagnosis device puts a subject whose driving ability is to be diagnosed (a driver who drives a car or the like) through a test (task) where the subject is made to touch a screen when a first mark is displayed on the screen, and to not touch the screen when a second mark is displayed on the screen. The driving ability diagnosis device performs diagnosis for a plurality of items regarding driving ability by one test, based on the number of times the first mark is correctly touched, the number of times the second mark is touched by mistake, time taken from display to touch, and the like. For example, the driving ability diagnosis device performs the test before the driver who is to drive a car or the like starts driving, for example, and performs diagnosis regarding the driving ability.

### (Example Configuration)

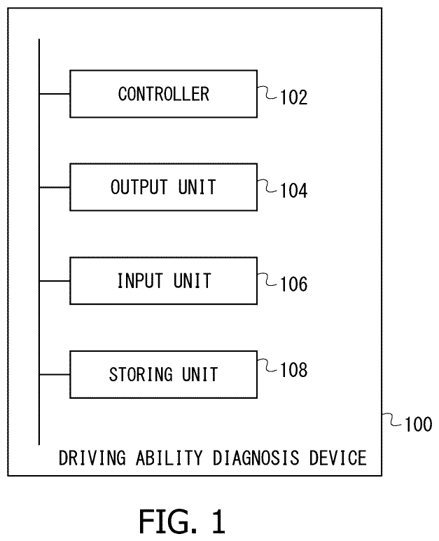

FIG. 1 is a diagram illustrating an example functional block of a driving ability diagnosis device 100 of the present embodiment. The driving ability diagnosis device 100 includes a controller 102, an output unit 104, an input unit 106, and a storing unit 108.

The controller 102 controls the entire driving ability diagnosis device 100. The controller 102 expands a program stored in the storing unit 108 in a work area in an executable form, and controls a peripheral appliance or the like through execution of the program, and thereby provides a function matching a predetermined object. Furthermore, the controller 102 performs various calculations.

The controller 102 controls a test to be taken by a user, based on a program stored in the storing unit 108, for example. In the test, the controller 102 performs a predetermined display on a display device of the output unit 104, and receives input by the user by an input device of the input unit 106. The controller 102 stores a result of the test in the storing unit 108.

The controller 102 calculates scores of cognitive function items related to the driving ability based on the result of the test taken by the user. Items related to driving ability here are speed of cognition, speed of awareness, consideration, concentration, attention, and judgment. Brain functions necessary for safe driving are indicated by the six items. Details of each item will be given later.

The output unit 104 includes a display device that displays a screen where the test is to be performed, for example. The display device displays images and text information. For example, the display device is an organic electroluminescence (EL) panel or a liquid crystal display panel.

The input unit 106 includes an input device such as a touch panel. The touch panel is an input device that is provided on a surface (display surface) of the display device of the output unit 104. For example, the touch panel is a capacitive touch panel that detects a contact position based on a change in capacitance caused by a part of the body, such as a finger of the user, coming into contact with an operation surface. A part of the body is a part such as a finger of a hand, a palm of a hand, back of a hand, or the like. Furthermore, contact is not limited to direct contact of a part of the body of the user, but may include indirect contact performed using a stylus or the like held in the hand of the user. The touch panel is not limited to a capacitive touch panel, and may be touch panels according to other methods, such as an optical touch panel. A coordinate system is set on the display surface of the display device, and the touch panel outputs a contact position using coordinates on the coordinate system. Coordinates correspond to a position on the display surface. The input unit 106 may include an input device such as buttons or a keyboard separate from the display device, instead of the touch panel. Input (operation) may be performed by a touch operation on the touch panel, a pressing operation on a button or a certain key on the keyboard, or the like.

The storing unit 108 stores data, programs and the like to be used by the driving ability diagnosis device 100.

The driving ability diagnosis device 100 can be implemented by a dedicated or general-purpose computer, such as a personal computer (PC), a work station (WS), a smartphone, a tablet terminal or a personal digital assistant (PDA), or an electronic appliance where a computer is mounted.

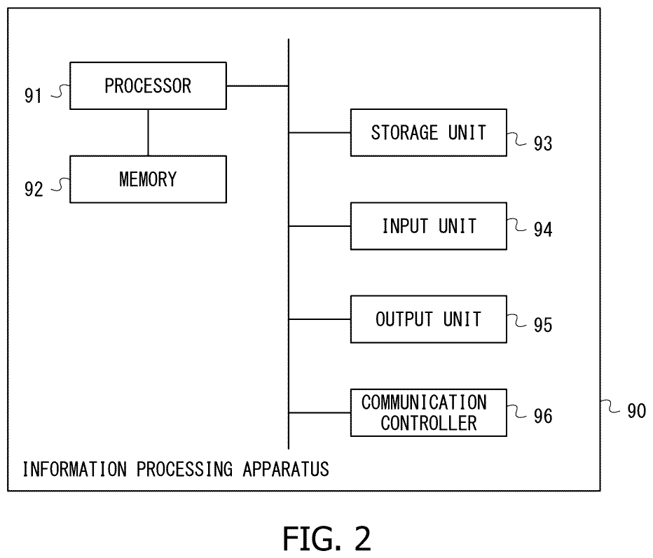

FIG. 2 is a diagram illustrating an example hardware configuration of an information processing apparatus. The information processing apparatus illustrated in FIG. 2 has a configuration of a general computer. The driving ability diagnosis device 100 is implemented by an information processing apparatus 90 as illustrated in FIG. 2. The information processing apparatus 90 in FIG. 2 includes a processor 91, a memory 92, a storage unit 93, an input unit 94, an output unit 95, and a communication controller 96. These are interconnected by a bus. The memory 92 and the storage unit 93 are computer-readable recording media. The hardware configuration of the computer is not limited to the example illustrated in FIG. 2, and structural elements may be omitted, substituted, or added as appropriate.

The processor 91 loads a program stored in a recording medium in a work area in the memory 92 and executes the program, and each structural component is controlled through execution of the program, and the information processing apparatus 90 can thus implement a function matching a predetermined object.

The processor 91 is a central processing unit (CPU) or a digital signal processor (DSP), for example.

The memory 92 includes a random access memory (RAM) and a read only memory (ROM), for example. The memory 92 is referred to also as a main memory.

The storage unit 93 is an erasable programmable ROM (EPROM) or a hard disk drive (HDD), for example. Furthermore, the storage unit 93 may include a removable medium, or in other words, a removable recording medium. The removable medium is a universal serial bus (USB) memory, or a disk recording medium such as a compact disc (CD) or a digital versatile disc (DVD), for example. The storage unit 93 is referred to also as a secondary memory.

The storage unit 93 stores various programs, various pieces of data, and various tables in a recording medium in a readable/writable manner. The storage unit 93 stores an operating system (OS), various programs, various tables, and the like. Information stored in the storage unit 93 may also be stored in the memory 92. Information stored in the memory 92 may also be stored in the storage unit 93.

The operating system is software that operates as an intermediary between software and hardware, and that manages a memory space, files, processes and tasks. The operating system includes a communication interface. The communication interface is a program for exchanging data with other external devices and the like connected via the communication controller 96. The external devices and the like include other computers and external memory, for example.

The input unit 94 includes a keyboard, a pointing device, a wireless remote control, or a touch panel. Furthermore, the input unit 94 may include an input device for videos and images, such as a camera, and an input device for sound, such as a microphone.

The output unit 95 includes a display device such as a liquid crystal display (LCD), an electroluminescence (EL) panel, a cathode ray tube (CRT) display or a plasma display panel (PDP), or an output device such as a printer. Furthermore, the output unit 95 may include an output device for sound, such as a speaker.

The communication controller 96 connects to another device, and controls communication between the information processing apparatus 90 and the device. For example, the communication controller 96 is a local area network (LAN) interface board, a wireless communication circuit for wireless communication, or a communication circuit for wired communication. The LAN interface board or the wireless communication circuit is connected to a network such as the Internet.

The computer that realizes the driving ability diagnosis device 100 achieves functions of the controller 102, the output unit 104, and the input unit 106 due to the processor loading, in the main memory, a program stored in an auxiliary memory and executing the program. The storing unit 108 is provided in a memory area of the main memory or the auxiliary memory.

### (Example Operation)

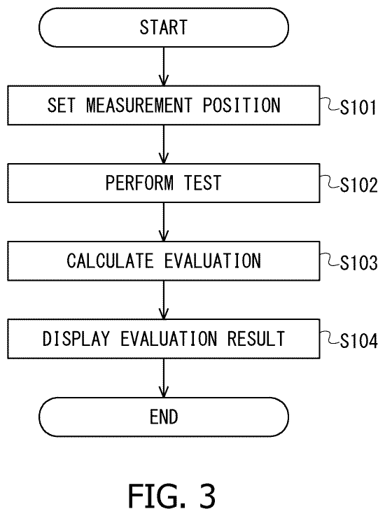

FIG. 3 is a diagram illustrating an example operation of the driving ability diagnosis device 100 according to the present embodiment. The driving ability diagnosis device 100 performs diagnosis related to a plurality of items related to driving ability of a user, according to a driving ability diagnosis application that is installed in advance. The controller 102 of the driving ability diagnosis device 100 starts a process in FIG. 3 when power of the driving ability diagnosis device 100 is turned on or when a driver performs a start operation on the driving ability diagnosis device 100.

In S101, the controller 102 adjusts a position of a head of a user such that a predetermined region on a screen (display screen) of the display device of the output unit 104 is made a central viewing field. The central viewing field here is a range of 5-degree viewing angle. The controller 102 displays a screen at the start of a test on the display device of the output unit 104.

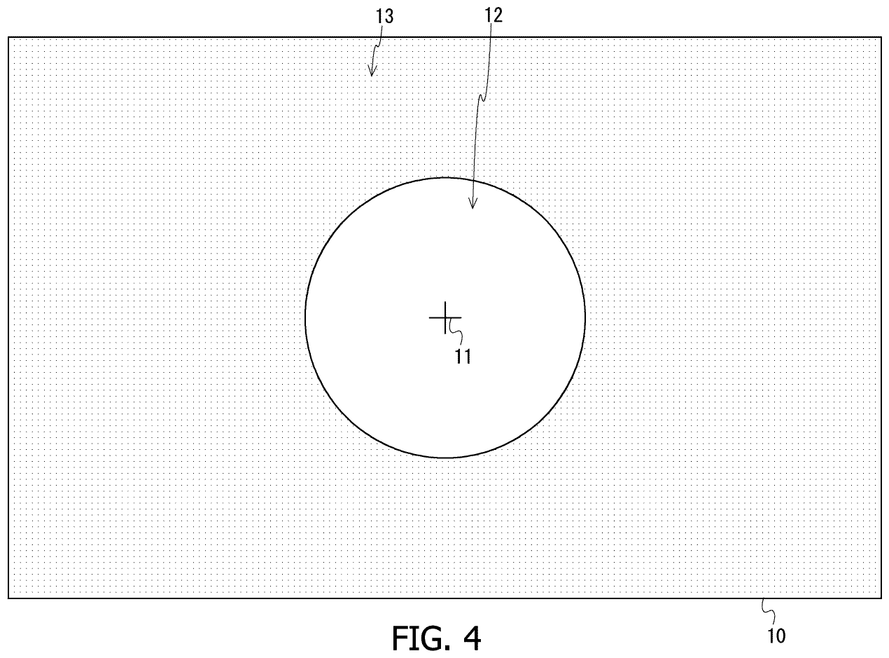

FIG. 4 is a diagram illustrating an example of a screen 10 at the time of start of a test by the driving ability diagnosis device 100. In the example in FIG. 4, the screen 10 is a horizontally long rectangle. A “+” mark 11 indicating a center position on the screen 10 is displayed at a center of the screen 10 at the time of start of the test. Moreover, a central region 12 that is a white circle centered on the center position on the screen 10 is displayed. The center position on the screen 10 is an intersection point of diagonal lines of the screen 10. A diameter of the circle of the central region 12 is about half the vertical length of the screen 10, but is not limited thereto. Moreover, a part outside the central region 12 is displayed in gray. The part indicated in gray will be referred to as a peripheral region 13.

The controller 102 performs output to urge a user to adjust a position of the head such that the central region 12 (predetermined region) is made the central viewing field of the user. That is, the controller 102 issues a notice so as to make the user adjust the position of the head such that an angle formed by a line segment connecting one end of a certain diameter of the circle of the central region 12 and a right eye of the user and a line segment connecting another end of the diameter of the circle and the right eye of the user is five degrees (a left eye may also be used instead of the right eye). For example, the controller 102 displays “please place your head at a position where the viewing field on the diameter of the circle at the center is five degrees”. Furthermore, the controller 102 acquires an actual vertical length of the screen 10 in advance, and calculates the diameter of the circle based on the length. Moreover, the controller 102 calculates, based on the diameter, a distance between the display device and the eye of the user at which the viewing angle on the central region 12 is five degrees. The controller 102 may display a message instructing to urge the user to make the distance between the display device and the eye of the user equal to the calculated distance. The position of the head of the user is thus adjusted such that the central region 12 on the screen 10 is made the central viewing field (5-degree viewing angle) of the user.

In S102, the controller 102 makes the user (subject) take a test. In the test, the user is made to touch the screen when the first mark is displayed on the screen, and not to touch the screen when the second mark is displayed on the screen. A position to be touched may be any position on the screen. At the time of the test, the user is notified in advance to touch the screen when the first mark is displayed, while looking at the “+” mark 11 at the center. For example, the controller 102 displays an image of a button indicating start on the screen 10 of the display device of the output unit 104, and starts the test when an operation of touching the image of the button by the user is received on the touch panel of the input unit 106. After the test is started, the controller 102 displays the first mark or the second mark in the central region 12 or the peripheral region 13 on the screen 10 of the display device of the output unit 104. The first mark or the second mark is sufficiently smaller in size than the central region 12. A touch operation on the touch panel may be a pressing operation on a button or a certain key on the keyboard. The touch operation and the pressing operation are examples of input (operation).

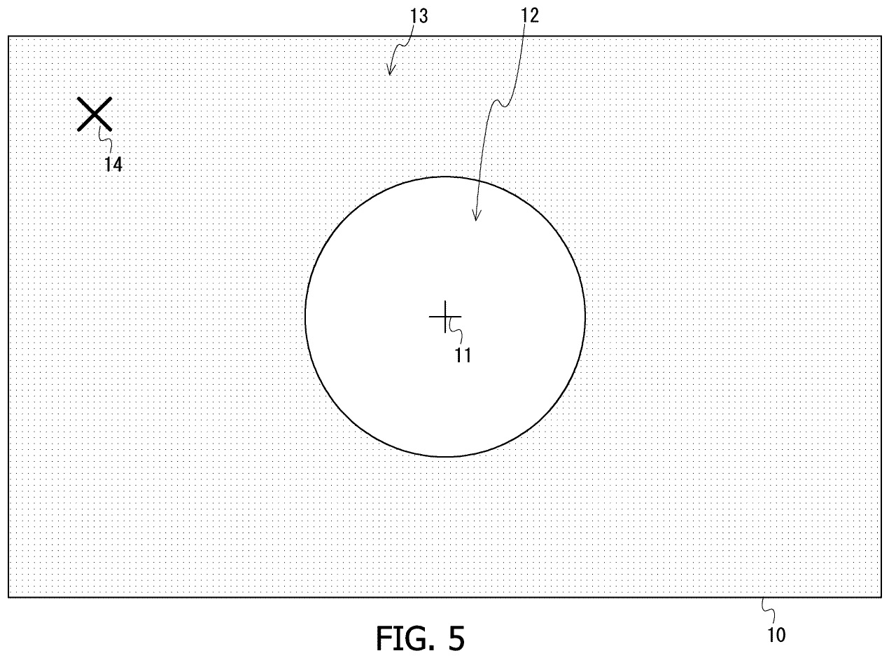

FIG. 5 is a diagram illustrating a first example of the screen 10 at the time of the test. In the example in FIG. 5, the central region 12 and the peripheral region 13, which are the same as those in FIG. 4, are displayed on the screen 10, and moreover, an “X” mark 14 is displayed in the peripheral region 13. The “X” mark 14 is displayed on an upper left side in the peripheral region 13. The “X”mark 14 is an example of the first mark.

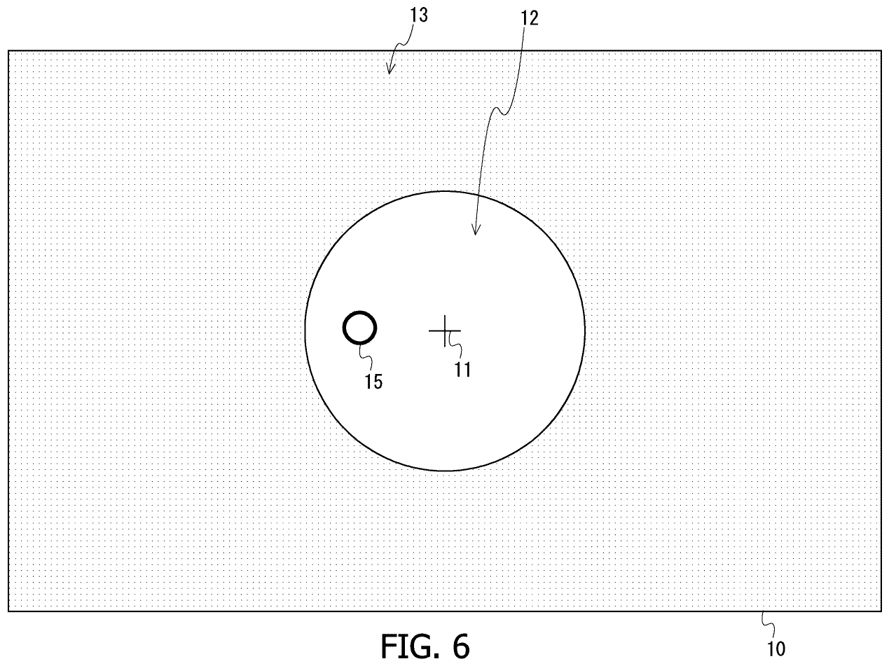

FIG. 6 is a diagram illustrating a second example of the screen 10 at the time of the test. In the example in FIG. 6, the central region 12 and the peripheral region 13, which are the same as those in FIG. 4, are displayed on the screen 10, and moreover, a “O” mark 15 is displayed in the central region 12. The “O” mark 15 is displayed on a left side in the central region 12. The “O” mark 15 is an example of the second mark.

As the first mark, a symbol, a letter, a picture or the like for suppressing an action of a person is used. Generally, the “X” mark 14 is an example of a symbol for suppressing an action of a person. Furthermore, as the second mark, a symbol, a letter, a picture or the like for encouraging an action of a person is used. Generally, the “O” mark 15 is an example of a symbol for prompting an action of a person. As the second mark, a symbol, a letter, a picture or the like for prompting an action of a person is used. By encouraging an action of the user when a symbol or the like for suppressing an action of a person is displayed, each item related to driving ability can be appropriately measured. The first mark and the second mark are different marks.

The controller 102 displays the “X” mark 14 or the “O” mark 15 in the central region 12 or the peripheral region 13 on the screen 10 of the display device of the output unit 104 for one second (predetermined period of time). Moreover, the controller 102 makes an interval between disappearance of the “X” mark 14 or the “O” mark 15 and next display of the “X” mark 14 or the “O” mark 15 a random interval of 0.5 seconds or longer and less than 1.5 seconds. The controller 102 judges whether a touch operation is received on the touch panel of the input unit 106 while the “X” mark 14 or the “O”mark 15 is displayed.

In the case where a touch operation is received on the touch panel of the input unit 106 while the “X” mark 14 is displayed, the controller 102 records a time (reaction time) from start of display of the “X” mark 14 to the touch operation, and stores the time in the storing unit 108 together with information about the position where the “X” mark 14 is displayed (or information about the region). A touch operation performed while the “X” mark 14 is displayed indicates that the touch operation is correctly performed. In the case where a touch operation is not received on the touch panel of the input unit 106 while the “X” mark 14 is displayed, the controller 102 records absence of a response to the “X” mark 14, and stores the information in the storing unit 108 together with the information about the position where the “X” mark 14 is displayed (or information about the region). That a touch operation is not performed while the “X” mark 14 is displayed indicates that the “X”mark 14 is overlooked.

In the case where a touch operation is received on the touch panel of the input unit 106 while the “O” mark 15 is displayed, the controller 102 records presence of a response to the “O” mark 15, and stores the information in the storing unit 108 together with information about the position where the “O” mark 15 is displayed (or information about the region). A touch operation performed while the “O” mark 15 is displayed indicates that the touch operation is performed by mistake. In the case where a touch operation is not received on the touch panel of the input unit 106 while the “O” mark 15 is displayed, the controller 102 records absence of a response to the “O” mark 15, and stores the information in the storing unit 108 together with the information about the position where the “O” mark 15 is displayed (or information about the region). That a touch operation is not performed while the “O” mark 15 is displayed indicates that it is correctly judged that a touch operation is not to be performed based on display of the “O”mark 15.

In one test, the controller 102 displays, in a random order, the “X” mark 14 and the “O” mark 15 in the central region 12 ten times and seven times, respectively, and the “X” mark 14 and the “O” mark 15 in the peripheral region 13 seven times and four times, respectively. Display positions of the “X” mark 14 or the “O” mark 15 in the central region 12 or the peripheral region 13 are randomly determined. The controller 102 may determine an approximate display region in advance so as to prevent a large bias in the display positions of the “X” mark 14 or the “O” mark 15 in the peripheral region 13 that may affect a measurement result. Because the “X” mark 14 and the “O” mark 15 that are displayed for one second are displayed 28 times in total, one at a time, at an interval of 0.5 seconds or longer and less than 1.5 seconds, one test takes about one minute. When all the “X” marks 14 and the “O” marks 15 have been displayed, one test is completed. The number of times and the regions of display of the “X” marks 14 and the “O” marks 15 in one test are not limited to those described above. When one test is completed, the process proceeds to S103.

In step S103, the controller 102 calculates scores for a plurality of items (evaluation items) related to driving ability based on a result of the test in S102 stored in the storing unit 108, and stores the scores in the storing unit 108. The items related to driving ability here are speed of cognition, speed of awareness, consideration, concentration, attention, and judgment. The score for each item is calculated on a 100-point scale. The controller 102 calculates the scores for the plurality of items based on the result of one test.

The speed of cognition is an item contributing to detection of a danger, a driving operation reaction, judgment, easy avoidance of a danger, and an appropriate action. For example, the speed of cognition is expressed by a speed of reaction to visual stimulation in the central viewing field. The score for the speed of cognition is determined by 214−0.26×(average value of reaction time (ms) to “X” mark 14 displayed in central region 12). The score for the speed of cognition is higher, the shorter the reaction time to the “X” mark 14 displayed in the central region 12. The speed of awareness is an item contributing to detection of a danger and a safe action. For example, the speed of awareness is expressed by a speed of reaction to visual stimulation in a peripheral viewing field (outside the central viewing field). The score for the speed of awareness is determined by 188−0.19×(average value of reaction time (ms) to “X” mark 14 displayed in peripheral region 13). The score for the speed of awareness is higher, the shorter the reaction time to the “X” mark 14 displayed in the peripheral region 13. Consideration is an item related to consideration to a peripheral part relative to a central part. For example, consideration is expressed by a relative speed of the speed of reaction to visual stimulation in the peripheral viewing field relative to the speed of reaction to visual stimulation in the central viewing field. The score for consideration is determined by 319−216×(average value of reaction time (ms) to “X” mark 14 displayed in peripheral region 13)/(average value of reaction time (ms) to “X”mark 14 displayed in central region 12).

Central concentration is an item related to cognition and capability to take an action in relation to a specific viewing field without being absent-minded or distracted. For example, central concentration is expressed by a low variation in the speed of reaction to visual stimulation in the central viewing field. The score for central concentration is determined by 123−361×(standard deviation of reaction time (ms) to “X” mark 14 displayed in central region 12)/(average value of reaction time (ms) to “X” mark 14 displayed in central region 12). The score for central concentration is higher, the lower the variation in the reaction time to the “X”mark 14 displayed in the central region 12.

Peripheral concentration is an item related to cognition and capability to take an action in relation to a wide viewing field without being absent-minded or distracted. For example, peripheral concentration is expressed by a low variation in the speed of reaction to visual stimulation in the peripheral viewing field. The score for peripheral concentration is determined by 123−361×(standard deviation of reaction time (ms) to “X” mark 14 displayed in peripheral region 13)/(average value of reaction time (ms) to “X” mark 14 displayed in peripheral region 13). The score for peripheral concentration is higher, the lower the variation in the reaction time to the “X” mark 14 displayed in the peripheral region 13. Additionally, in the present embodiment, central concentration and peripheral concentration are used as the evaluation items, but it is also possible to use only central concentration as the evaluation item related to concentration. Furthermore, an average of the score for central concentration and the score for peripheral concentration may be determined as the score for concentration.

Central attention relates to an ability to carefully and swiftly check every symbol appearing in a specific viewing field. The score for central attention is 100 points when the number of times of absence of response to the “X” mark 14 displayed in the central region 12 is zero, 75 points when the number of times is one, 50 points when the number of times is two, and 30 points when the number of times is three or more. Peripheral attention relates to an ability to carefully and swiftly check every symbol appearing in various positions in the viewing field. The score for peripheral attention is 100 points when the number of times of absence of response to the “X” mark 14 displayed in the peripheral region 13 is zero, 75 points when the number of times is one, 50 points when the number of times is two, and 30 points when the number of times is three or more. Additionally, in the present embodiment, central attention and peripheral attention are used as the evaluation items, but it is also possible to use only peripheral attention as the evaluation item related to attention. Furthermore, an average of the score for central attention and the score for peripheral attention may be determined as the score for attention.

Judgment is an item indicating smallness in the number of errors at the time of a driving operation. Judgment relates to an ability to take a swift action while using inhibition function among cognitive functions. The score for judgment is 100 points when the number of times of presence of response to the “O” mark 15 is zero, 75 points when the number of times is one, 50 points when the number of times is two, and 30 points when the number of times is three or more. Additionally, calculation regarding judgment may be performed by categorizing judgment into judgment in the central region (central judgment) and judgment in the peripheral region (peripheral judgment). For example, the score for central judgment is 100 points when the number of times of presence of response to the “O” mark in the central region is zero, 75 points when the number of times is one, 50 points when the number of times is two, and 30 points when the number of times is three or more. Furthermore, the score for peripheral judgment is 100 points when the number of times of presence of response to the “O” mark in the peripheral region is zero, 75 points when the number of times is one, 50 points when the number of times is two, and 30 points when the number of times is three or more.

In the case where the calculated score is 100 points or higher, the controller 102 may set that the score is 100 points. In the case where the calculated score is less than 30 points, the controller 102 may set that the score is 30 points. The method of calculating the score for each item is not limited to the method described above, and may be changed within a range based on property of each item. The controller 102 stores the calculated score for each item in the storing unit 108. The higher the score for each item, the higher the driving ability. For example, when the score for each item is 80 points or higher, it can be considered that it is safe (one can drive safely), and when the score is less than 50 points, it is considered that caution is needed (one cannot drive safely). The score for each item may change depending on health conditions of the user, and may be used for the purpose of managing health of the user. The controller 102 may perform calculation for at least two items among the six items related to the driving ability described above.

In S104, the controller 102 displays the calculated score for each item on the display device of the output unit 104. For example, the controller 102 displays the calculated score for each item by plotting the same on a radar chart. The user can check the score for each item related to the driving ability based on the display.

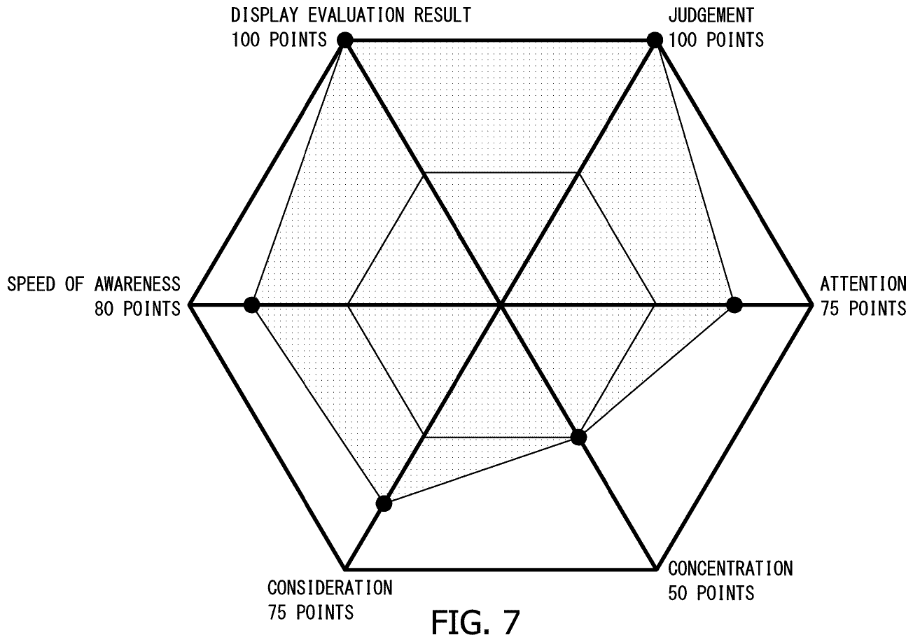

FIG. 7 is a diagram illustrating an example of a radar chart indicating a score for each item related to the driving ability. The radar chart in FIG. 7 indicates the scores for the items related to the driving ability, where the items are the speed of cognition, the speed of awareness, consideration, concentration, attention, and judgment. The radar chart indicates that the greater the area of the hexagon, the higher the driving ability.

### (Others)

Calculation of the score for each item in S103 may be performed by another information processing apparatus that is communicatively connected to the driving ability diagnosis device 100. In this case, the driving ability diagnosis device 100 transmits the result of the test to another information processing apparatus, and the other information processing apparatus calculates the score for each item based on the result of the test. The other information processing apparatus transmits the calculated score for each item to the driving ability diagnosis device 100.

### (Operations and Effects of Embodiment)

The Driving ability diagnosis device 100 makes a driver (user) who drives a car or the like to take a test for diagnosing the driving ability. In the test, one is to touch the screen when the first mark is displayed on the screen, and not to touch the screen when the second mark is displayed on the screen. The driving ability diagnosis device 100 calculates scores for a plurality of items related to the driving ability based on the reaction time, in the test, between display of the first mark and a touch, for example. With the driving ability diagnosis device 100, the scores for a plurality of items related to the driving ability can be calculated by combining an average value of reaction time in each region, a standard deviation of reaction time, and the like obtained by one test. With the driving ability diagnosis device 100, the scores for a plurality of items related to the driving ability can be calculated by one test that takes about one minute. With the driving ability diagnosis device 100, a plurality of items related to the driving ability can be easily diagnosed. With the driving ability diagnosis device 100, diagnosis can be easily and frequently performed in relation to a plurality of items related to the driving ability of a driver. With the driving ability diagnosis device 100, a change in the driving ability of a driver can be easily grasped by frequently performing the test.

### Second Embodiment

In the first embodiment described above, the driving ability diagnosis device 100 calculates the scores for a plurality of items, and plots and displays the score for each item on a radar chart. In a present embodiment, a driving ability diagnosis device 100A further estimates and outputs a character of a driver based on the scores for a plurality of items. The present embodiment has a same configuration as the first embodiment except that a character is estimated and output, and a redundant description will be omitted by denoting a same element by a same reference sign.

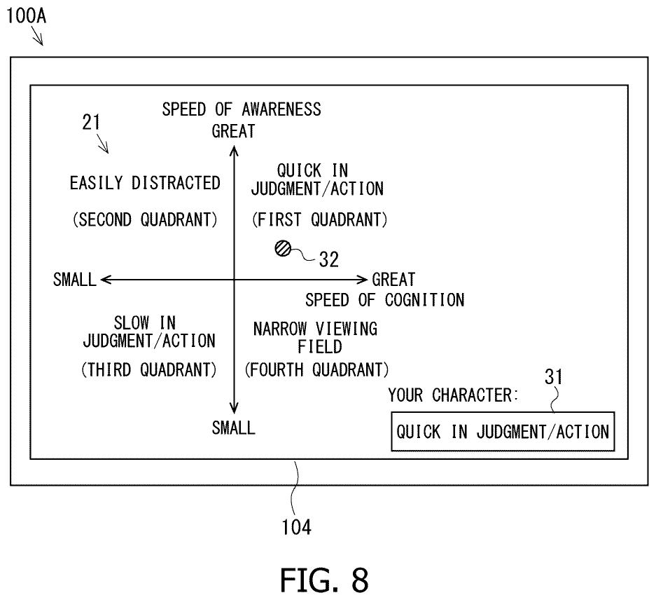

FIG. 8 is a diagram illustrating an example where the driving ability diagnosis device 100A according to the present embodiment displays a character of a driver on the display device of the output unit 104. A two-dimensional chart 21 where a vertical axis and a horizontal axis represent two selected items, and a character 31 of a driver are displayed. Additionally, the two-dimensional chart 21, and a method of generating the character 31 of a driver will be described later.

Like that of the driving ability diagnosis device 100 illustrated in FIG. 1, a functional block of the driving ability diagnosis device 100A includes the controller 102, the output unit 104, the input unit 106, and the storing unit 108. A hardware configuration of the driving ability diagnosis device 100A is the same as the one illustrated in FIG. 2.

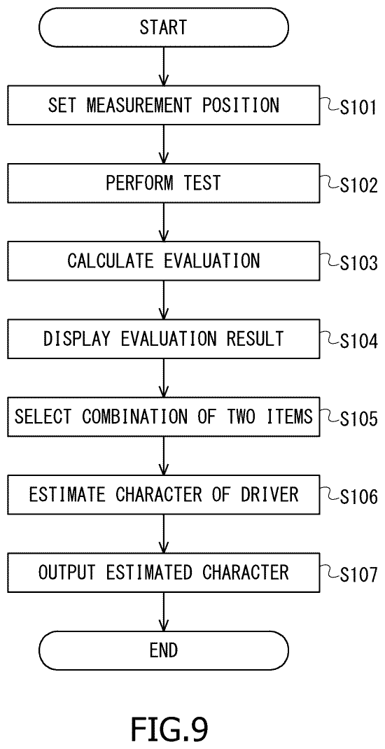

FIG. 9 is a diagram illustrating an example operation of the driving ability diagnosis device 100A according to the present embodiment. Additionally, the processes from S101 to S104 in FIG. 9 are the same as those in FIG. 3.

In S105, the controller 102 selects a combination of two items from the plurality of items. For example, the controller 102 reads, from the storing unit 108, setting information indicating a predetermined combination of items, such as “speed of cognition” and “speed of awareness” or “speed of cognition” and “central attention”, and selects a combination of two items based on the setting information. The setting information is stored in the storing unit 108 in advance. The controller 102 may alternatively select a combination of two items based on selection by a user. For example, the controller 102 displays a plurality of items on the display device of the output unit 104 and asks a driver to perform selection, and selects two items selected by the driver as items to be used for estimation of a character at a later stage. In this case, the controller 102 may display, as options, items such as “speed of cognition”, “speed of awareness”, “consideration”, “central concentration”, “peripheral concentration”, “central attention”, “peripheral attention”, “central judgment”, and “peripheral judgment”, and may make the driver select any two of the items. The controller 102 may also select all the combinations of two items using all the items for which the scores are calculated in S103. That is, if the number of items for which the score is calculated in S103 is N, the number m of all the combinations is N×(N−1)/2.

In S106, the controller 102 acquires the scores calculated in S103 from the storing unit 108 in relation to the items selected in S105, and estimates the character of the driver based on the scores. For example, the controller 102 refers to a character data table used for estimation of a character, acquires character information corresponding to the scores, and estimates the character of the driver to be the character indicated by the character information.

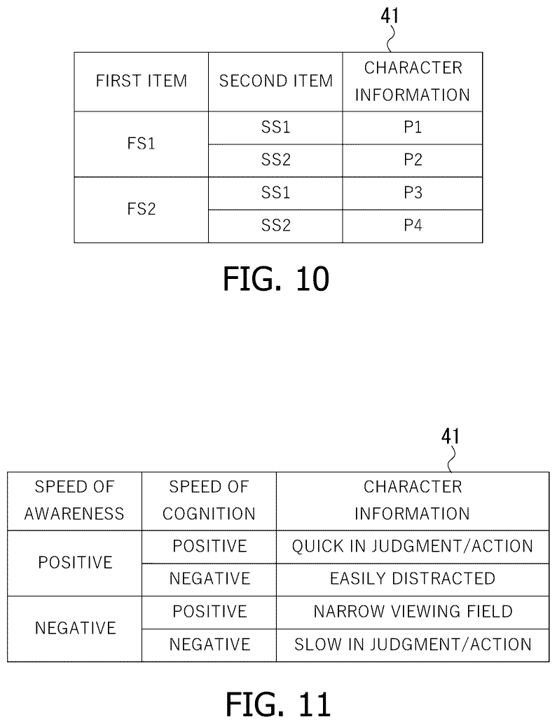

FIG. 10 is a diagram illustrating an example of a character data table 41. In the character data table 41 in FIG. 10, the score for a first item is classified into two categories, namely, FS1 and FS2, and the score for a second item is classified into two categories, namely, SS1 and SS2. That is, there are four combinations regarding the categories related to the first item and the categories related to the second item. In other words, in the character data table 41, the scores FS1 and FS2 for the first item are each classified into the scores SS1 and SS2 for the second item to obtain four categories. In the character data table 41, character information P1, . . . , P4 is registered in association with each combination of a category related to the first item and a category related to the second item. For example, in the case where the score related to the first item is FS1 and the score related to the second item is SS1, the controller 102 acquires the character information P1 that is associated with such a combination from the character data table 41, and estimates that the character of the driver is the character indicated by the character information P1. For example, in the case where the score related to the first item is FS2 and the score related to the second item is SS1, the controller 102 acquires the character information P3 that is associated with such a combination from the character data table 41, and estimates that the character of the driver is the character indicated by the character information P3.

FIG. 11 is a diagram illustrating a specific example of the character data table 41. In the character data table 41 in FIG. 11, “speed of awareness” is selected as the first item, and “speed of cognition” is selected as the second item. Furthermore, in the character data table 41 in FIG. 11, each of the score for “speed of awareness” and the score for “speed of cognition” is Z-scored and classified into two values, namely, a positive value and a negative value. Additionally, an average value to be used at the time of Z-scoring is determined in advance by averaging test results (scores for respective items) for a large number of drivers. In the case where the test is taken by a driver himself/herself a plurality of times in the past, it is also possible to use a value that is obtained by averaging results of the test taken a plurality of times. In the character data table 41 in FIG. 11, character information such as “quick in judgment/action”, “easily distracted”, “narrow viewing field”, or “slow in judgment/action” is registered in association with each combination of scores. For example, in the case where the scores related to “speed of awareness” and “speed of cognition” are both positive, the controller 102 acquires the character information, “quick in judgment/action”, that is associated with such a combination, and estimates that the driver is quick in judgment/action. For example, in the case where the score related to “speed of awareness” is negative and the score related to “speed of cognition” is positive, the controller 102 acquires the character information, “narrow viewing field”, that is associated with such a combination from the character data table 41, and estimates that the driver has a narrow viewing field.

Additionally, categories in the character data table 41 in FIG. 11 are merely examples, and the categories are not limited to the example illustrated in FIG. 11. For example, positive and negative values of the scores for respective items may each be classified into several stages according to σ. Furthermore, the score for each item may be classified into positive and negative values by setting a mode value or a median value to zero (origin), without using Z-scoring. Also in this case, the positive and negative values may be classified into a plurality of categories.

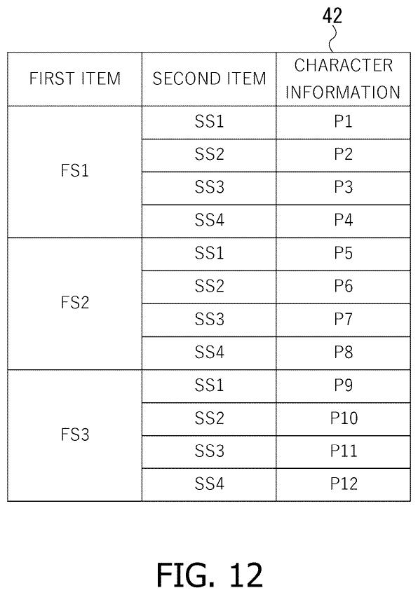

FIG. 12 is a diagram illustrating an example of a character data table 42 where scores for items are classified into a different number of categories than in FIG. 10. In the character data table 42 in FIG. 12, the score for the first item is classified into three categories, namely, FS1, FS2 and FS3, and the score for the second item is classified into four categories, namely, SS1 to SS4. That is, there are 12 combinations regarding the categories related to the first item and the categories related to the second item. Moreover, in the character data table 42, character information P1, . . . , P12 is registered in association with each combination of a category related to the first item and a category related to the second item. In this manner, the score for the first item and the score for the second item may be classified into a plurality of categories, and may each be classified into three or more categories, for example. Moreover, the score for the first item and the score for the second item may be classified into different numbers of categories.

Additionally, the character data tables 41, 42 are created in advance for each combination of items that can be selected in S105, and are stored in the storing unit 108. For example, in the case where there are N items from which selection is to be performed, the number m of all the combinations is N×(N−1)/2 as described above, and m character data tables 41, 42 are created. Furthermore, the method for estimating the character of a user from the scores related to two selected items may use an artificial intelligence (AI) model instead of the character data table. In this case, for example, a large number of combinations of a result (score for each item) of the test taken by a user (driver) and a correct answer label indicating the character of the user are prepared, and an AI model (machine learning model) is created by performing machine-learning using such combinations as training data. The controller 102 may input the scores for selected items to the AI model, and may acquire the character of the user from the AI model.

In S107, the controller 102 displays the character estimated in S106 on the display device of the output unit 104. For example, as illustrated in FIG. 8, the controller 102 displays the two-dimensional chart 21 and the character 31 of the driver. The two-dimensional chart 21 in FIG. 8 is a graph that takes “speed of awareness” (first item) as the vertical axis, and “speed of cognition” (second item) as the horizontal axis. In the two-dimensional chart 21, an average value of a data set accumulating the test results for a large number of drivers is made an origin of each axis such that the test result (score for each item) is in a normal distribution. That is, in the two-dimensional chart 21, the origin of the vertical axis is an average value of “speed of awareness” in the data set, and the origin of the horizontal axis is an average value of “speed of cognition” in the data set. Furthermore, the controller 102 converts the score calculated in S103 to a value that takes the average value of the data set to be zero. For example, the controller 102 Z-scores the scores for “speed of awareness” and “speed of cognition” calculated in S103 (hereinafter referred to also as “subject score”) by the following formula using the data set.

Z score=(subject score−average value of data set)/standard deviation

In the following, the Z-score is also referred to as a converted score.

Alternatively, the controller 102 may obtain a value (converted score) that is obtained by subtracting the average value of the data set from the subject score. For example, in the case where the average value of the data set is 90, the converted score obtained by subtracting 90 is 10 if the subject score is 100, and the converted score obtained by subtracting 90 is zero if the subject score is 90. Furthermore, in the case where the subject score is 65, the converted score after subtraction of 90 is −25. In this manner, when the subject score is greater than the average value, the converted score takes a positive value, and when the subject score is smaller than the average value, the converted score takes a negative value. Additionally, the value to be the origin in relation to the subject score (hereinafter referred to also as “reference value”) is not limited to the average value, and may instead be a mode value or a median value.

The controller 102 plots a mark 32 at a position corresponding to the converted score for each item on the two-dimensional chart 21. In the example in FIG. 8, the converted scores for “speed of awareness” and “speed of cognition” both take positive values, and the mark 32 is plotted in a first quadrant on the upper right. In this case, the controller 102 refers to the character data table 41 in FIG. 11, and acquires the character information “quick in judgment/action” associated with the converted scores. The controller 102 displays character information 23 on the display device of the output unit 104 in the manner as illustrated in FIG. 8, and presents the character to the driver (subject). Additionally, the character information 23 may also be output as an audio message from a speaker of the output unit 104.

In the two-dimensional chart 21 in FIG. 8, if the converted score for “speed of awareness” is positive and the converted score for “speed of cognition” is negative, the mark 32 is plotted in a second quadrant on the upper left. In this case, the controller 102 refers to the character data table 41 in FIG. 11, acquires the character information “easily distracted” associated with the converted scores, and displays the same on the display device. In the same manner, in the case where the converted scores for “speed of awareness” and “speed of cognition” are both negative, the mark 32 is plotted in a third quadrant on the lower left. In this case, the controller 102 refers to the character data table 41 in FIG. 11, acquires the character information “slow in judgment/action” associated with the converted scores, and displays the same on the display device. Furthermore, in the case where the converted score for “speed of awareness” is negative and the converted score for “speed of cognition” is positive, the mark 32 is plotted in a fourth quadrant on the lower right. In this case, the controller 102 refers to the character data table 41 in FIG. 11, acquires the character information “narrow viewing field” associated with the converted scores, and displays the same on the display device.

In the example in FIG. 8, the controller 102 displays the character information acquired from the character data table 41 on the display device as it is, but the character information may instead be displayed in combination with other information pieces. For example, additional information such as “extremely”, “rather”, or “slightly more than average” may be displayed in combination with the character information according to a level of deviation of the score for each item from the average value (reference value). For example, in the case where the character information acquired from the character data table 41 is “slow in judgment/action”, “extremely slow in judgment/action”, “rather slow in judgment/action”, or “slightly slower in judgment/action than average” may be displayed. Furthermore, comment information taking character into consideration may be registered in advance in the character data table 41, and the controller 102 may acquire the comment information from the character data table 41 together with the character information, and display the same. Moreover, the controller 102 may acquire the test results for a large number of drivers and determine an average value or a representative value such as a mode value or a median value for each age, determine an ability age based on which of the representative values for ages the score of the driver who is the subject is closest to, and display the ability age together with the character information.

Additionally, in the example in FIG. 8, the item “speed of awareness” is assigned to the vertical axis, and the item “speed of cognition” is assigned to the horizontal axis, but such an example is not restrictive, and the item “speed of cognition” may be assigned to the vertical axis, and the item “speed of awareness” may be assigned to the horizontal axis. Moreover, each of the two items selected in S105 may be assigned to an axis (horizontal axis or vertical axis) specified by the driver. Moreover, which of two items in a combination is to be assigned to which of the vertical axis and the horizontal axis may be determined in advance for each combination of items and be stored in the storing unit 108 as setting information. In this case, the controller 102 reads out the setting information from the storing unit 108 according to the combination of two items selected in S105, and assigns each item to the vertical axis or the horizontal axis according to the setting information.

In the two-dimensional chart 21 in FIG. 8, the greater the value on the vertical axis, the greater the “speed of awareness”, and the smaller the value on the vertical axis, the smaller the “speed of awareness”. Moreover, in the two-dimensional chart 21, the greater the value on the horizontal axis, the greater the “speed of cognition”, and the smaller the value on the horizontal axis, the smaller the “speed of cognition”. However, such an example is not restrictive, and a relationship between the value on each axis and a change in each item (“speed of awareness” and “speed of cognition”) may be opposite the example in FIG. 8.

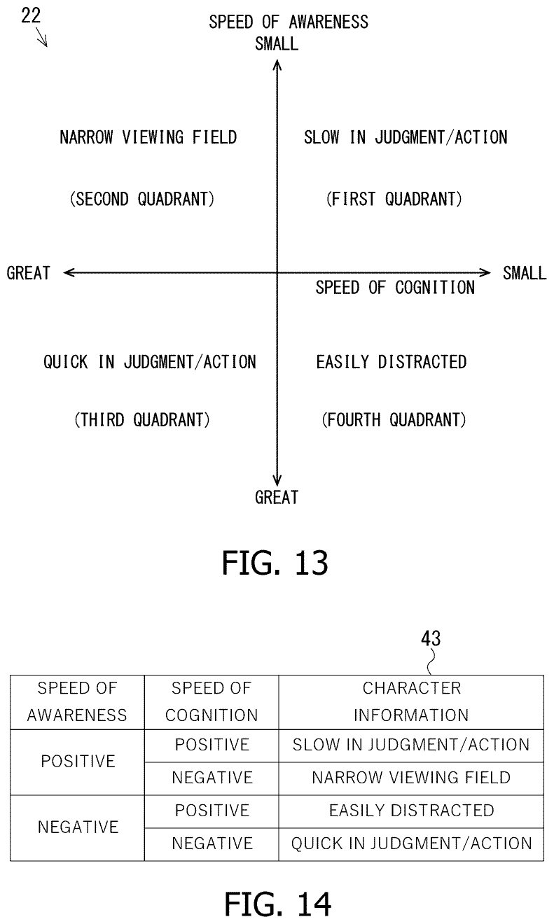

FIG. 13 is a diagram illustrating an example of a two-dimensional chart 22 where a relationship between a value on each axis and a change in each item is different than in FIG. 8. Additionally, the example in FIG. 13 is different from the example of the two-dimensional chart 21 in FIG. 8 with respect to the relationship between the value on each axis and a change in each item, but is the same in other respects. In the two-dimensional chart 22 in FIG. 13, the greater the value on the vertical axis (the higher in the drawing), the smaller the “speed of awareness”, and the smaller the value on the vertical axis (the lower in the drawing), the greater the “speed of awareness”. Furthermore, in the two-dimensional chart 22 in FIG. 13, the greater the value on the horizontal axis (more to the right in the drawing), the smaller the “speed of cognition”, and the smaller the value on the horizontal axis (more to the left in the drawing), the greater the “speed of cognition”.

In the case of using the two-dimensional chart 22 in FIG. 13, the controller 102 converts the score calculated in S103 while taking the average value (reference value) of the data set as zero, multiplies the converted score by −1, and inverts positive/negative sign of the converted score. In the following, the converted score whose positive/negative sign is inverted will also be referred to as an inverted score. Furthermore, in this case, a character data table according to the inverted score is created in advance. FIG. 14 is a diagram illustrating a specific example of a character data table 43 created according to inverted scores. In the character data table 43 in FIG. 14, “speed of awareness” is selected as the first item, and “speed of cognition” is selected as the second item. Furthermore, in the character data table 43 in FIG. 14, the inverted score for “speed of awareness” and the inverted score for “speed of cognition” are each classified into two categories, namely, a positive value and a negative value. In the character data table 43 in FIG. 14, “slow in judgment/action” is associated with a case where the inverted scores related to “speed of awareness” and “speed of cognition” are both positive. “Easily distracted” is associated with a case where the inverted score related to “speed of awareness” is negative and the inverted score related to “speed of cognition” is positive, and “narrow viewing field” is associated with a case where the inverted score related to “speed of awareness” is positive and the inverted score related to “speed of cognition” is negative. Moreover, “quick in judgment/action” is associated with a case where the inverted scores related to “speed of awareness” and “speed of cognition” are both negative. The controller 102 uses the character data table 43, and can acquire the character information corresponding to the inverted scores and estimate the character of the driver in the same manner as in the example in FIG. 11 described above. For example, in the case where the inverted score related to “speed of awareness” is negative and the inverted score related to “speed of cognition” is positive in S106, the controller 102 plots the mark 32 in a fourth quadrant of the two-dimensional chart 22, and acquires the character information, “easily distracted”, corresponding to the inverted scores from the character data table 43, and displays the same on the display device.

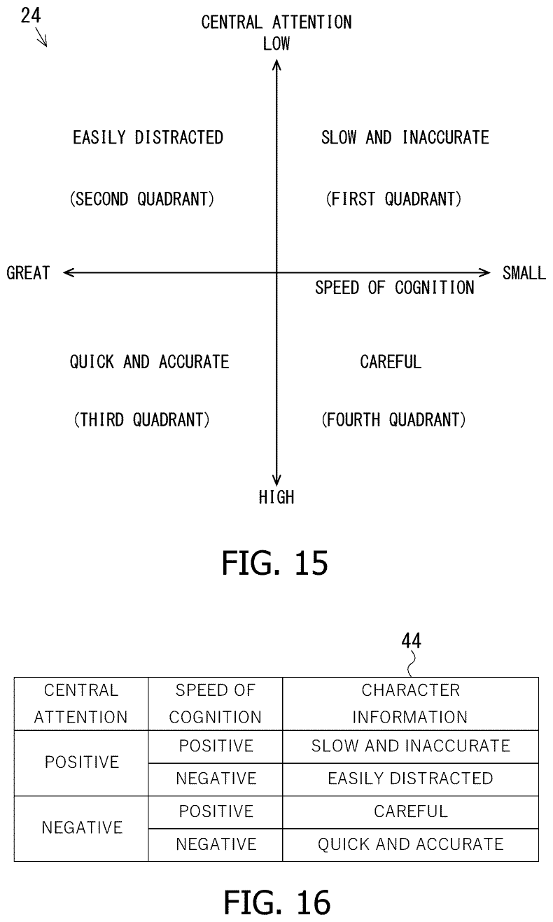

FIG. 15 is a diagram illustrating an example where “central attention” and “speed of cognition” are selected as a combination of two items. Additionally, the example in FIG. 15 is different from the example in FIG. 13 with respect to a selected item, but is the same in other respects. In a two-dimensional chart 24 in FIG. 15, the greater the value on the vertical axis, the lower the “central attention”, and the smaller the value on the vertical axis, the higher the “central attention”. Furthermore, in the two-dimensional chart 24 in FIG. 15, the greater the value on the horizontal axis, the smaller the “speed of cognition”, and the smaller the value on the horizontal axis, the greater the “speed of cognition”. In this case, the controller 102 converts the score calculated in S103 while taking the average value (reference value) of the data set as zero, inverts positive/negative sign of the converted score, and determines the inverted score.

FIG. 16 is a diagram illustrating a specific example of a character data table 44 where “central attention” and “speed of cognition” are selected and scores are inverted. In the character data table 44 in FIG. 16, “central attention” is selected as the first item, and “speed of cognition” is selected as the second item. Furthermore, in the character data table 44 in FIG. 16, the inverted score for “central attention” and the inverted score for “speed of cognition” are each classified into two categories, namely, a positive value and a negative value. In the character data table 44 in FIG. 16, “slow and inaccurate” is associated with a case where the inverted scores related to “central attention” and “speed of cognition” are both positive. “Careful” is associated with a case where the inverted score related to “central attention” is negative and the inverted score related to “speed of cognition” is positive, and “easily distracted” is associated with a case where the inverted score related to “central attention” is positive and the inverted score related to “speed of cognition” is negative. Moreover, “quick and accurate” is associated with a case where the inverted scores related to “central attention”and “speed of cognition”are both negative. The controller 102 uses the character data table 44, and can acquire the character information corresponding to the inverted scores and estimate the character of the driver in the same manner as in the example in FIG. 11 described above. For example, in the case where the inverted score related to “central attention” is negative and the inverted score related to “speed of cognition” is negative in S106, the controller 102 plots the mark 32 in a third quadrant of the two-dimensional chart 24, and acquires the character information, “quick and accurate”, corresponding to the inverted scores from the character data table 44, and displays the same on the display device.

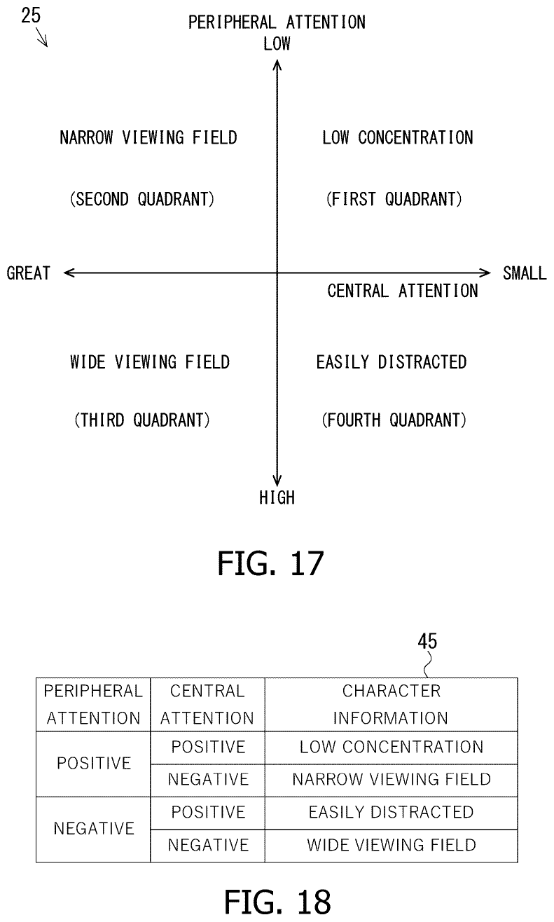

FIG. 17 is a diagram illustrating an example where “peripheral attention” and “central attention” are selected as a combination of two items. Additionally, the example in FIG. 17 is different from the example in FIG. 13 with respect to selected items, but is the same in other respects. In a two-dimensional chart 25 in FIG. 17, the greater the value on the vertical axis, the smaller the “peripheral attention”, and the smaller the value on the vertical axis, the greater the “peripheral attention”. Furthermore, in the two-dimensional chart 25 in FIG. 17, the greater the value on the horizontal axis, the smaller the “central attention”, and the smaller the value on the horizontal axis, the greater the “central attention”. In this case, the controller 102 converts the score calculated in S103 while taking the average value (reference value) of the data set as zero, inverts positive/negative sign of the converted score, and determines the inverted score.

FIG. 18 is a diagram illustrating a specific example of a character data table 45 where “peripheral attention” and “central attention” are selected and scores are inverted. In the character data table 45 in FIG. 18, “peripheral attention” is selected as the first item, and “central attention” is selected as the second item. Furthermore, in the character data table 45 in FIG. 18, the inverted score for “peripheral attention” and the inverted score for “central attention” are each classified into two categories, namely, a positive value and a negative value. In the character data table 45 in FIG. 18, “low concentration” is associated with a case where the inverted scores related to “peripheral attention” and “central attention” are both positive. “Easily distracted” is associated with a case where the inverted score related to “peripheral attention” is negative and the inverted score related to “central attention” is positive, and “narrow viewing field” is associated with a case where the inverted score related to “peripheral attention” is positive and the inverted score related to “central attention” is negative. Moreover, “wide viewing field” is associated with a case where the inverted scores related to “peripheral attention” and “central attention” are both negative. The controller 102 uses the character data table 45, and can acquire the character information corresponding to the inverted scores and estimate the character of the driver in the same manner as in the example in FIG. 11 described above. For example, in the case where the inverted score related to “peripheral attention” is positive and the inverted score related to “central attention” is positive in S106, the controller 102 plots the mark 32 in a first quadrant of the two-dimensional chart 25, and acquires the character information, “low concentration”, corresponding to the inverted scores from the character data table 45, and displays the same on the display device.

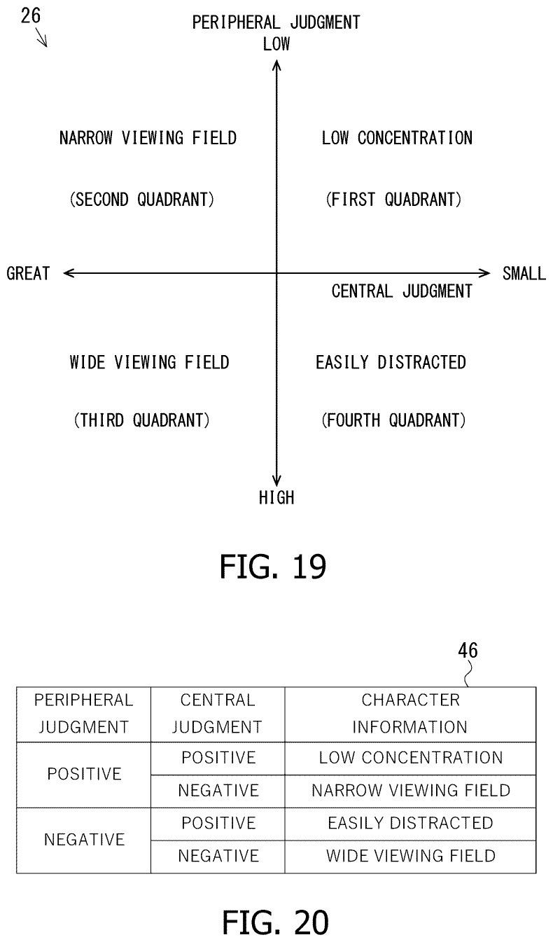

FIG. 19 is a diagram illustrating an example where “peripheral judgment” and “central judgment” are selected as a combination of two items. Additionally, the example in FIG. 19 is different from the example in FIG. 13 with respect to selected items, but is the same in other respects. In a two-dimensional chart 26 in FIG. 19, the greater the value on the vertical axis, the lower the “peripheral judgment”, and the smaller the value on the vertical axis, the higher the “peripheral judgment”. Furthermore, in the two-dimensional chart 26 in FIG. 19, the greater the value on the horizontal axis, the lower the “central judgment”, and the smaller the value on the horizontal axis, the higher the “central judgment”. In this case, the controller 102 converts the score calculated in S103 while taking the average value (reference value) of the data set as zero, inverts positive/negative sign of the converted score, and determines the inverted score.

FIG. 20 is a diagram illustrating a specific example of a character data table 46 where “peripheral judgment” and “central judgment” are selected and scores are inverted. In the character data table 46 in FIG. 20, “peripheral judgment” is selected as the first item, and “central judgment” is selected as the second item. Furthermore, in the character data table 46 in FIG. 20, the inverted score for “peripheral judgment” and the inverted score for “central judgment” are each classified into two categories, namely, a positive value and a negative value. In the character data table 46 in FIG. 20, “low concentration” is associated with a case where the inverted scores related to “peripheral judgment” and “central judgment” are both positive. “Easily distracted” is associated with a case where the inverted score related to “peripheral judgment” is negative and the inverted score related to “central judgment” is positive, and “narrow viewing field” is associated with a case where the inverted score related to “peripheral judgment” is positive and the inverted score related to “central judgment” is negative. Moreover, “wide viewing field” is associated with a case where the inverted scores related to “peripheral judgment”and “central judgment”are both negative. The controller 102 uses the character data table 46, and can acquire the character information corresponding to the inverted scores and estimate the character of the driver in the same manner as in the example in FIG. 11 described above. For example, in the case where the inverted score related to “peripheral judgment” is positive and the inverted score related to “central judgment” is negative in S106, the controller 102 plots the mark 32 in a second quadrant of the two-dimensional chart 26, and acquires the character information, “narrow viewing field”, corresponding to the inverted scores from the character data table 46, and displays the same on the display device.

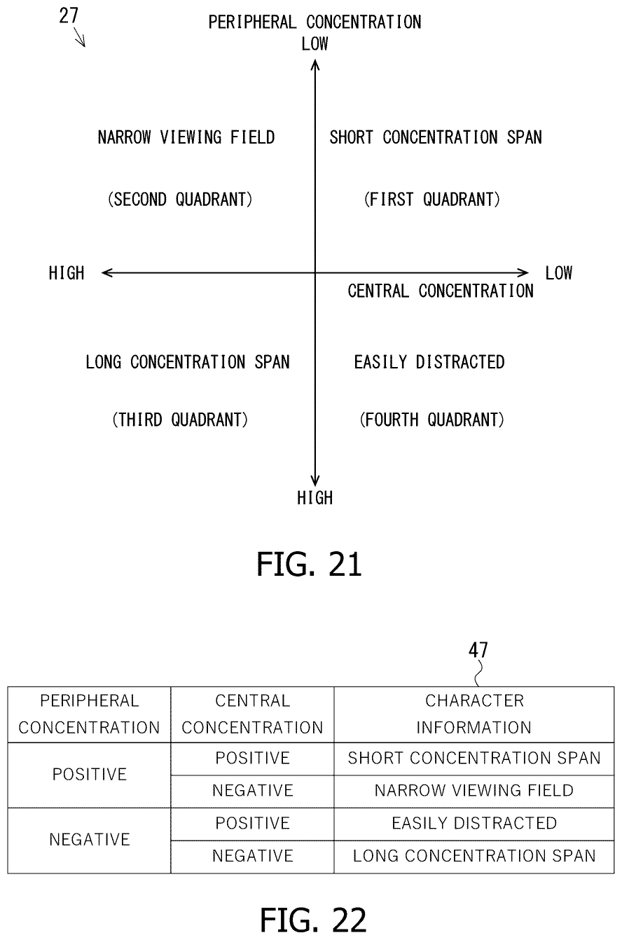

FIG. 21 is a diagram illustrating an example where “peripheral concentration” and “central concentration” are selected as a combination of two items. Additionally, the example in FIG. 21 is different from the example in FIG. 13 with respect to selected items, but is the same in other respects. In a two-dimensional chart 27 in FIG. 21, the greater the value on the vertical axis, the lower the “peripheral concentration”, and the smaller the value on the vertical axis, the higher the “peripheral concentration”. Furthermore, in the two-dimensional chart 27 in FIG. 21, the greater the value on the horizontal axis, the lower the “central concentration”, and the smaller the value on the horizontal axis, the higher the “central concentration”. In this case, the controller 102 converts the score calculated in S103 while taking the average value (reference value) of the data set as zero, inverts positive/negative sign of the converted score, and determines the inverted score.

FIG. 22 is a diagram illustrating a specific example of a character data table 47 where “peripheral concentration” and “central concentration” are selected and scores are inverted. In the character data table 47 in FIG. 22, “peripheral concentration” is selected as the first item, and “central concentration” is selected as the second item. Furthermore, in the character data table 47 in FIG. 22, the inverted score for “peripheral concentration” and the inverted score for “central concentration” are each classified into two categories, namely, a positive value and a negative value. In the character data table 47 in FIG. 22, “short concentration span” is associated with a case where the inverted scores related to “peripheral concentration” and “central concentration” are both positive. “Easily distracted” is associated with a case where the inverted score related to “peripheral concentration” is negative and the inverted score related to “central concentration” is positive, and “narrow viewing field” is associated with a case where the inverted score related to “peripheral concentration” is positive and the inverted score related to “central concentration” is negative. Moreover, “long concentration span” is associated with a case where the inverted scores related to “peripheral concentration” and “central concentration” are both negative. The controller 102 uses the character data table 47, and can acquire the character information corresponding to the inverted scores and estimate the character of the driver in the same manner as in the example in FIG. 11 described above. For example, in the case where the inverted score related to “peripheral concentration” is positive and the inverted score related to “central concentration” is positive in S106, the controller 102 plots the mark 32 in a first quadrant of the two-dimensional chart 27, and acquires the character information, “short concentration span”, corresponding to the inverted scores from the character data table 47, and displays the same on the display device. As described above, in the present embodiment, description has been given, with reference to FIGS. 13 to 22, on examples regarding a combination of two items selected from a plurality of items and estimation of the character of the driver based on the scores for the selected items. Also when items other than those in FIGS. 13 to 22 are selected, the driving ability diagnosis device 100A of the present embodiment is able to estimate and output the character of the driver based on the scores for the selected items in the same manner as in FIGS. 13 to 22. In this case, also in relation to items other than those in FIGS. 13 to 22, the driving ability diagnosis device 100A registers, in the character data table 41 in FIG. 10, categories of the score related to the first item and categories of the score related to the second item, and registers character information P1, . . . , P4 in association with each combination, and stores the character data table 41 in the storing unit 108. That is, the registered character data table 41 is created for each combination of all the items that may be selected in S105, and is stored in the storing unit 108. Accordingly, also in a case where items other than those in FIGS. 13 to 22 are selected, the controller 102 can estimate the character of the driver in the same manner as in the examples in FIGS. 13 to 22 by using the registered character data table 41.

### (Operations and Effects of Embodiment)

The driving ability diagnosis device 100A makes a driver (user) who drives a car or the like to take a test for diagnosing the driving ability, and calculates scores for a plurality of items related to driving ability based on a reaction time or the like of the driver taking the test. With the driving ability diagnosis device 100A, scores for a plurality of items related to driving ability can be calculated by combining average value of reaction time in each region, standard deviation of reaction time and the like obtained in one test, and estimate the character of the driver based on the scores for two items among the plurality of items. With the driving ability diagnosis device 100A, scores for a plurality of items related to driving ability can be calculated by one test that takes about one minute, and the character of the driver can be estimated from multiple perspectives. The driving ability diagnosis device 100A allows the driver to grasp his/her character, by outputting the estimated character to the driver.

Components described above may be combined in any way possible. In the embodiments described above, the driving ability diagnosis devices 100, 100A display the score for each item related to driving ability and the character of the driver obtained as a result of a test on the display device, but the result of the test may be transmitted to an information terminal carried by the driver or another system so as to be used by the information terminal or the system.

### Computer-Readable Recording Medium

A program for causing a computer, a machine, or a device (hereinafter “computer or the like”) to achieve any of the functions described above can be recorded in a recording medium that can be read by the computer or the like. The function can be provided when the computer or the like reads and executes the program in the recording medium.

Here, the recording medium that can be read by the computer or the like refers to a recording medium that accumulates information such as data or programs by an electrical, magnetic, optical, mechanical, or chemical action in a manner allowing reading by the computer or the like. Elements that constitute a computer, such as a CPU and a memory, may be provided in such a recording medium to cause the CPU to execute the program.

Among such recording media, those that are removable from the computer or the like are, for example, a flexible disk, a magneto-optical disk, a CD-ROM, a CD-R/W, a DVD, a DAT, an 8 mm tape, and a memory card.

Furthermore, examples of the recording media that are installed in the computer or the like include a hard disk and a ROM.

### REFERENCE SIGNS LIST

10: screen
11: “+” mark
12: central region
13: peripheral region
14: “X” mark
15: “O” mark
100, 100A: driving ability diagnosis device
102: controller
104: output unit
106: input unit
108: storing unit
90: information processing apparatus
91: processor
92: memory
93: storage unit
94: input unit
95: output unit
96: communication controller

What is claimed is:

1. A driving ability diagnosis device comprising:
an output unit comprised to display a screen including a central region including a center position and a peripheral region other than the central region;
an input unit comprised to receive input from a user; and
a controller comprised to cause the output unit to display a first mark or a second mark different from the first mark in the central region or the peripheral region a predetermined number of times for a predetermined period of time, judge whether input from the user is received by the input unit while the first mark or the second mark is displayed, measure a reaction time that is a period of time from when the first mark is displayed to when the input unit receives input from the user, in a case where the first mark is displayed, and calculate scores for a plurality of items related to driving ability of the user, based on whether input from the user is received by the input unit while the first mark or the second mark is displayed and the reaction time.

2. The driving ability diagnosis device according to claim 1, wherein the plurality of items include speed of cognition that is based on an average value of the reaction time to the first mark displayed in the central region.

3. The driving ability diagnosis device according to claim 1, wherein the plurality of items include speed of awareness that is based on an average value of the reaction time to the first mark displayed in the peripheral region.

4. The driving ability diagnosis device according to claim 1, wherein the plurality of items include consideration that is based on a ratio between an average value of the reaction time to the first mark displayed in the central region and an average value of the reaction time to the first mark displayed in the peripheral region.

5. The driving ability diagnosis device according to claim 1, wherein the plurality of items include concentration that is based on a standard deviation of the reaction time to the first mark displayed in the central region.

6. The driving ability diagnosis device according to claim 1, wherein the plurality of items include attention that is based on number of times that a touch operation by the user is not received by the input unit while the first mark is displayed in the peripheral region.

7. The driving ability diagnosis device according to claim 1, wherein the plurality of items include judgment that is based on number of times that a touch operation by the user is received by the input unit while the second mark is displayed in the peripheral region.

8. The driving ability diagnosis device according to claim 1, wherein the controller selects two items from the plurality of items, and estimates character of the user based on the scores for the two items selected.

9. The driving ability diagnosis device according to claim 8, wherein the plurality of items include at least one of
speed of cognition that is based on an average value of the reaction time to the first mark displayed in the central region,
speed of awareness that is based on an average value of the reaction time to the first mark displayed in the peripheral region,
consideration that is based on a ratio between the average value of the reaction time to the first mark displayed in the central region and the average value of the reaction time to the first mark displayed in the peripheral region,
central concentration that is based on a standard deviation of the reaction time to the first mark displayed in the central region,
peripheral concentration that is based on a standard deviation of the reaction time to the first mark displayed in the peripheral region,
central attention that is based on number of times that a touch operation by the user is not received by the input unit while the first mark is displayed in the central region,
peripheral attention that is based on number of times that a touch operation by the user is not received by the input unit while the first mark is displayed in the peripheral region,
central judgment that is based on number of times that a touch operation by the user is received by the input unit while the second mark is displayed in the central region, and
peripheral judgment that is based on number of times that a touch operation by the user is received by the input unit while the second mark is displayed in the peripheral region.

10. The driving ability diagnosis device according to claim 8, wherein a character data table where character information indicating character is registered in association with a score for a first item that is one of the plurality of items and a score for a second item, among the plurality of items, different from the first item is referred to, and the character is estimated from the character information corresponding to scores for two items calculated based on input from the user.

11. A driving ability diagnosis method performed by a computer, the method comprising:
displaying a screen including a central region including a center position and a peripheral region other than the central region;
receiving input from a user; and
displaying a first mark or a second mark different from the first mark in the central region or the peripheral region a predetermined number of times for a predetermined period of time, judging whether input from the user is received while the first mark or the second mark is displayed, measuring a reaction time that is a period of time from when the first mark is displayed to when input from the user is received, in a case where the first mark is displayed, and calculating scores for a plurality of items related to driving ability of the user, based on whether input from the user is received while the first mark or the second mark is displayed and the reaction time.

12. The driving ability diagnosis method according to claim 11, wherein the computer selects two items from the plurality of items, and estimates character of the user based on the scores for the two items selected.

13. A non-transitory storage medium storing a driving ability diagnosis program for causing a computer to:
display a screen including a central region including a center position and a peripheral region other than the central region,
receive input from a user, and
display a first mark or a second mark different from the first mark in the central region or the peripheral region a predetermined number of times for a predetermined period of time, judge whether input from the user is received while the first mark or the second mark is displayed, measure a reaction time that is a period of time from when the first mark is displayed to when input from the user is received, in a case where the first mark is displayed, and calculate scores for a plurality of items related to driving ability of the user, based on whether input from the user is received while the first mark or the second mark is displayed and the reaction time.

14. The non-transitory storage medium storing the driving ability diagnosis program according to claim 13, wherein the computer is caused to select two items from the plurality of items, and estimate character of the user based on scores for the two items.
